SEQUENCE LISTING (3 sequences)

SEQ ID NO: 1 - protein, 28 aa, Artificial Sequence
  synthetic peptide
TLKERCLQVVRSLVKKKRTLRKNDRKKR

SEQ ID NO: 2 - protein, 13 aa, Artificial Sequence
  synthetic peptide
KKRTLRKNDRKKR

SEQ ID NO: 3 - protein, 22 aa, Artificial Sequence
  synthetic peptide
  MOD_RES (2)..(2): aminohexanoic acid
  MOD_RES (5)..(5): bAla
  MOD_RES (8)..(8): aminohexanoic acid
  MOD_RES (16)..(16): aminohexanoic acid
  MOD_RES (18)..(18): bAla
  MOD_RES (20)..(20): aminohexanoic acid
  MOD_RES (22)..(22): bAla
RXRRARRXRYQFLIRXRARXRA

(19) United States
(12) Patent Application Publication
(10) Pub. No.: US 2022/0008547 A1
(43) Pub. Date: Jan. 13, 2022

(54) Carrier Peptide Fragment and use Thereof

(71) Applicant: TOAGOSEI CO., LTD, Tokyo (JP)

(72) Inventors: Nahoko Baileykobayashi, Tsukuba-Shi (JP); Tetsuhiko Yoshida, Tsukuba-Shi (JP)

(21) Appl. No.: 17/361,498

(22) Filed: Jun. 29, 2021

(30) Foreign Application Priority Data
Jul. 7, 2020 (JP) 2020-117211

(51) Int. Cl. A61K 47/64; C07K 2/00

(52) U.S. Cl. CPC A61K 47/6455; A61K 38/00; C07K 2/00

(57) ABSTRACT

A method of introducing a foreign substance from the outside of eukaryotic cells into at least a cytoplasm of the cells in vitro or in vivo disclosed here includes the following steps:
(1) preparing a construct for introducing a foreign substance, wherein the construct includes a carrier peptide fragment composed of the following amino acid sequence:
TLKERCLQVVRSLVKKKRTLRKNDRKKR (SEQ ID NO: 1), and
the foreign substance bound to an N-terminal side and/or C-terminal side of the carrier peptide fragment;
(2) supplying the construct into a sample containing target eukaryotic cells; and
(3) incubating the sample to which the construct is supplied, wherein the construct is introduced into the target eukaryotic cells in the sample.

### CROSS-REFERENCE TO RELATED APPLICATIONS

Priority is claimed on Japanese Patent Application No. 2020-117211, filed Jul. 7, 2020, the content of which is incorporated herein by reference.

### TECHNICAL FIELD

The present invention relates to a carrier peptide fragment and use thereof, and specifically, to introducing a foreign substance into eukaryotic cells using a carrier peptide fragment composed of an amino acid sequence shown in SEQ ID NO: 1.

### TECHNICAL BACKGROUND

In the related art, a technology in which a foreign substance such as a polypeptide and a nucleic acid, particularly, a biologically active substance, is introduced into cells (eukaryotic cells) of humans and other mammals has been used. In such a technology, the traits of cells into which a foreign substance is introduced (furthermore, tissues and organs composed of the cells) are changed and their functions are enhanced and improved.

Regarding a method of introducing a foreign substance such as a polypeptide and a nucleic acid into eukaryotic cells, for example, as described in WO 2011/013700 (Patent Document 1) and The Journal of Clinical Investigation, Vol. 129, 2019, pp. 4739-4744, a cell-penetrating peptide may be used (Non-patent Document 1). In Patent Document 1, a carrier peptide fragment and a biologically active substance are bonded to form a construct for introducing a foreign substance. When such a construct is supplied to eukaryotic cells (stem cells), a biologically active substance is introduced into cells. Further, it is described in this patent literature that the eukaryotic cells can be induced to differentiate. Non-patent Document 1 discloses a cell-penetrating peptide having a configuration different from that of Patent Document 1. It has been described that a conjugate of such a cell-penetrating peptide and an oligonucleotide can be used as a therapeutic agent for myotonic dystrophy.

### SUMMARY OF THE INVENTION

In recent years, there has been an increasing interest in cell-penetrating peptides. Thus, it is desired to develop a technology for improving the efficiency of introducing a foreign substance into desired cells compared to the related art.

The present invention has been made in view of such circumstances, and an object (objective) of the present invention is to provide a technology for more efficiently introducing a desired foreign substance into the cytoplasm of eukaryotic cells.

The inventors conducted screening in order to evaluate cell permeability of a synthetic peptide having various amino acid sequences. Then, they found an amino acid sequence having excellent cell permeability and which can be preferably used as a carrier peptide (carrier peptide fragment), and completed the present invention.

That is, according to the technology disclosed here, a method of introducing (transferring) a foreign substance from the outside (that is, outside the cell membrane) of eukaryotic cells (particularly, various animal cells having no cell wall represented by humans and other mammals) into at least the cytoplasm of the cells in vitro or in vivo is provided.

The method includes the following steps:

(1) preparing a construct for introducing a foreign substance, wherein the construct includes a carrier peptide fragment composed of the following amino acid sequence:

TLKERCLQVVRSLVKKKRTLRKNDRKKR (SEQ ID NO: 1), and the foreign substance bound to an N-terminal side and/or C-terminal side of the carrier peptide fragment;

(2) supplying the construct into a sample containing target eukaryotic cells; and

(3) incubating the sample to which the construct is supplied, wherein the construct is introduced into the target eukaryotic cells in the sample.

Here, the “foreign substance” is an inorganic compound or an organic compound that can be directly bound to the N-terminal side or C-terminal side of the carrier peptide fragment or can be indirectly bound thereto via an appropriate linker, and has a molecular size and chemical properties with which it can be introduced into eukaryotic cells.

According to the above configuration, when a construct for introducing a foreign substance constructed by directly bonding a desired foreign substance to an N-terminal side and/or C-terminal side of the carrier peptide fragment or by indirectly bonding thereto via an appropriate linker is supplied (that is, added to viable eukaryotic cells) into a sample containing desired eukaryotic cells (typically, a culture containing the cells), the desired foreign substance can be efficiently introduced from the outside of eukaryotic cells (outside the cell membrane) into the cytoplasm through the cell membrane.

In a preferred aspect, the foreign substance is any organic compound selected from the group consisting of polypeptides, nucleic acids, dyes and drugs.

Here, the “polypeptide” refers to a polymer having a structure in which a plurality of amino acids are bonded by peptide bonds. The polypeptide is not limited by the number of peptide bonds (that is, the number of amino acid residues). That is, the polypeptides include those generally called peptides having about 10 or more and less than 300 amino acid residues, and those generally called proteins (typically, polymer compounds composed of 300 or more amino acid residues). In this field, polypeptides and proteins are not strictly classified. In this specification, polymers (including oligomers) composed of a plurality of amino acid residues are collectively referred to as polypeptides.

In addition, the “nucleic acid” refers to a polymer of nucleotides and includes DNA and RNA. The “nucleic acid” is not limited by the number of bases.

In a preferred aspect, the foreign substance is a mature polypeptide derived from any biological species or its precursor polypeptide, and the construct for introducing a foreign substance is a synthetic polypeptide having an amino acid sequence corresponding to a mature polypeptide or its precursor polypeptide as the foreign substance and an amino acid sequence of the carrier peptide fragment.

According to such a configuration, a synthetic peptide having an amino acid sequence of the mature polypeptide or its precursor polypeptide and an amino acid sequence of the carrier peptide fragment can be introduced into desired eukaryotic cells.

Preferably, the amino acid sequence corresponding to a mature polypeptide or its precursor polypeptide as the foreign substance is arranged on the N-terminal side of the carrier peptide fragment.

According to such a configuration, the amino acid sequence of the mature polypeptide or its precursor polypeptide can be introduced into desired eukaryotic cells more efficiently.

In addition, in a preferred aspect, target eukaryotic cells to which the construct for introducing a foreign substance is introduced are human or non-human mammal cells.

Such a configuration is suitable for introducing a foreign substance into human or non-human mammal cells.

In addition, according to the technology disclosed here, there is provided a construct for introducing a foreign substance produced in order to introduce a foreign substance from the outside of eukaryotic cells into at least a cytoplasm of the cells.

The construct for introducing a foreign substance includes a carrier peptide fragment composed of the following amino acid sequence:

TLKERCLQVVRSLVKKKRTLRKNDRKKR (SEQ ID NO: 1), and the foreign substance bound to an N-terminal side and/or C-terminal side of the carrier peptide fragment.

When the construct for introducing a foreign substance having such a configuration is used, a desired foreign substance can be efficiently introduced into desired eukaryotic cells.

### BRIEF DESCRIPTION OF THE DRAWINGS

FIG. 1 is a histogram obtained by analysis using a flow cytometer in Example 2 and in which the X axis represents fluorescent intensity, and in which the Y axis represents the number of cells.

### DESCRIPTION OF THE RELATED EMBODIMENTS

Preferable embodiments of the present invention will be described below. Components other than those particularly mentioned in this specification (for example, the primary structure and chain length of carrier peptide fragments) that are necessary for implementation of the present invention (for example, a method of chemically synthesizing peptides, a cell culture technique, and a general method of preparing a composition including peptides and nucleic acids as components) can be recognized by those skilled in the art as design matters based on the related art in the fields of cell engineering, physiology, medicine, pharmacy, organic chemistry, biochemistry, genetic engineering, protein engineering, molecular biology, genetics, and the like. The present invention can be implemented based on content disclosed in this specification and common general technical knowledge in the field. Here, in the following description, amino acids are represented by one-letter symbols (but, three-letter symbols in the sequence listing).

The entire content of all documents cited in this specification is incorporated herein by reference.

In addition, the “synthetic peptide” in this specification refers to a peptide fragment of which a peptide chain alone is not independently and stably present in nature, but it is produced through artificial chemical synthesis or biosynthesis (that is, production based on genetic engineering) and can be stably present in a predetermined composition. Here, the term “peptide” refers to an amino acid polymer having a plurality of peptide bonds, and is not limited by the number of amino acid residues.

In addition, the term “amino acid residue” in this specification includes an N-terminal amino acid and a C-terminal amino acid of a peptide chain unless otherwise specified.

Here, always, the left side of the amino acid sequence described in this specification is the N terminal side, and the right side thereof is the C terminal side.

A construct for introducing a foreign substance disclosed here includes a carrier peptide fragment composed of the following amino acid sequence:

TLKERCLQVVRSLVKKKRTLRKNDRKKR (SEQ ID NO: 1), and a foreign substance bound to an N-terminal side and/or C-terminal side of the carrier peptide fragment.

The “carrier peptide fragment” is a sequence defined (determined) by an amino acid sequence shown in SEQ ID NO: 1, and is an amino acid sequence that exhibits cell permeability with respect to eukaryotic cells.

As described above, the inventors conducted screening and found that the amino acid sequence shown in SEQ ID NO: 1 is a carrier peptide fragment having excellent cell permeability. In addition, as shown in the following examples, the inventors found through this examination that such amino acids have superior cell permeability to conventional cell-penetrating peptide sequences (refer to Patent Document 1, Non-patent Document 1 and JOURNAL OF BIOLOGICAL CHEMISTRY, Vol. 281, No. 35, 2006, pp. 25223-25230 (Non-patent Document 2)).

The amino acid sequence shown in SEQ ID NO: 1 is an amino acid sequence composed of 15 consecutive amino acid residues from the 157^{th} to 171^{st} amino acid sequence of a VHL (Von Hippel-Lindau) protein and an amino acid sequence in which an amino acid sequence composed of a total of 13 amino acid residues from the 491^{st} amino acid residue to 503^{rd} amino acid residue of LIM kinase 2 (LIM Kinase 2) is directly bonded.

Here, it is known that the VHL protein exhibits neural differentiation induction (PNAS, Vol. 95, 1998, pp. 114-119 (Non-patent Document 3)). In addition, it is known that the VHL protein is a kind of protein kinase involved in LIM kinase 2 intracellular signal transduction and is present in human endothelial cells.

It is known that an amino acid sequence composed of 13 amino acid residues of the LIM kinase 2 is NoLS (Patent Document 1 and Non-patent Document 2). Therefore, the amino acid sequence shown in SEQ ID NO: 1 can introduce a foreign substance into at least the cytoplasm of cells from the outside. Such an amino acid sequence can additionally introduce a foreign substance into the nucleus (inside the nuclear membrane).

The “carrier peptide fragment” is typically the same amino acid sequence as the amino acid sequence shown in SEQ ID NO: 1. The “carrier peptide fragment” includes a modified sequence of such an amino acid sequence as long as the cell permeability is not impaired. Here, the “modified sequence” is an amino acid sequence (modified amino acid sequence) formed by substituting, deleting and/or adding (inserting) one or several (typically, 2 or 3) amino acid residues. Slightly modified sequences are included in the “carrier peptide fragment” as a technical idea disclosed here because they can be easily used by those skilled in the art based on information disclosed here.

Typical examples of a modified sequence in this specification include, for example, a sequence formed by a so-called conservative amino acid replacement in which 1, 2, or 3 amino acid residues are conservatively substituted, and a sequence in which 1, 2, or 3 amino acid residues are added (inserted) or deleted with respect to a predetermined amino acid sequence. Typical examples of conservative amino acid replacement include, for example, a sequence in which a basic amino acid residue is replaced with another basic amino acid residue (for example, mutual substitution between a lysine residue and an arginine residue) and a sequence in which a hydrophobic amino acid residue is replaced with another hydrophobic amino acid residue (for example, a mutual substitution between a leucine residue, an isoleucine residue, and a valine residue).

The construct for introducing a foreign substance disclosed here can be designed and constructed by directly bonding (linking) a desired foreign substance to an N-terminal side and/or C-terminal side of the carrier peptide fragment or indirectly bonding (linking) via an appropriate linker.

The linker is not particularly limited, but may be a peptide linker or a non-peptide linker. Although not particularly limited, the amino acid sequence constituting a peptide linker is preferably an amino acid sequence that does not cause steric hindrance and is flexible. The peptide linker can be, for example, a linker composed of 10 or less (more preferably, 1 or more and 5 or less, for example, 1, 2, 3, 4, or 5 amino acid residues) amino acid residues containing 1 or 2 or more amino acid residues selected from among glycine, alanine, and serine. In addition, β-alanine may be used as such a linker. The non-peptide linker is not particularly limited, and for example, an alkyl linker, a polyethylene glycol (PEG) linker, an aminohexanoyl spacer or the like may be used.

The foreign substance is typically an organic compound such as a polypeptide, a nucleic acid, a dye, or a drug.

The foreign substance can be, for example, a polypeptide. When the foreign substance is a polypeptide, a peptide chain is designed so that it includes an amino acid sequence constituting the polypeptide and an amino acid sequence constituting a carrier peptide fragment, the peptide chain is biosynthesized or chemically synthesized, and thereby a construct for introducing a desired foreign substance can be produced.

In addition, the foreign substance may be an organic compound, for example, nucleic acids such as various DNA or RNA, dyes (for example, various fluorescent dye compounds such as FITC), or drugs (for example, antitumor agents including a nucleic acid-based antitumor agent such as 5-fluorouracil (5FU) and antiviral agents such as azidothymidine (AZT)).

As described above, the foreign substance has various functions in introduction target eukaryotic cells. Although not particularly limited, functions of the foreign substance may include, for example, promotion of stem cell differentiation induction (stem cell differentiation inducing activity), suppression of tumor cell proliferation (antitumor activity), and suppression of virus-infected cell proliferation (antiviral activity).

According to various conventionally known chemical methods, such an organic compound can be directly bonded to an N-terminal side and/or C-terminal side of the carrier peptide fragment or indirectly bonding thereto to construct a construct for introducing a foreign substance.

The number of foreign substances that bind to the carrier peptide fragment is not particularly limited. That is, one or more foreign substances may be bound to one carrier peptide fragment. Although not particularly limited, for example, a polypeptide, a nucleic acid, a drug or the like may be bound to an N-terminal side of one carrier peptide fragment, and a dye may be bound to a C-terminal side. Binding a dye to the carrier peptide fragment is preferable in consideration of evaluating introduction efficiency of the construct for introducing a foreign substance.

Here, when the foreign substance is a polypeptide, the polypeptide to be adopted (amino acid sequence) is not particularly limited. For example, a substance having a relatively large number of amino acid residues such as a polypeptide or protein having about 100 to 1,000 amino acid residues can be used as the foreign substance.

Typically, the synthetic peptide produced as a construct for introducing a foreign substance has a total number of amino acid residues of several to several tens (for example, 10) or more. The total number of amino acid residues is appropriately 1,000 or less, preferably 600 or less, more preferably 500 or less, and particularly preferably 300 or less (for example, 10 to 300). Polypeptides with such a length are easily synthesized (biosynthesis, chemical synthesis) and easily used.

Regarding the foreign substance, a mature type or a precursor (including a pro-type and a pre-pro type) of a polypeptide involved in functions such as development, differentiation, proliferation, canceration, homeostasis, metabolic regulation, and the like of various cells and tissues (organs) is preferable. In addition, the present invention can be implemented to clarify functions of polypeptides in cells (in living tissues) by introducing polypeptides whose functions are not known in the related art into the cells.

For example, when eukaryotic cells into which a foreign substance is introduced are stem cells of humans and other mammals, it is preferable to use a mature type or a precursor of a polypeptide having various physiological activities involved in induction of differentiation of the stem cells. Here, the “stem cells” include somatic stem cells, embryonic stem cells, and induced pluripotent stem cells (hereinafter referred to as iPS cells). In addition, when eukaryotic cells into which a foreign substance is introduced are cancer cells (tumor cells), it is preferable to use various polypeptides involved in induction of apoptosis of the cancer cells (tumor cells). Alternatively, in this case, it is preferable to use a polypeptide that can prevent cancer cells (tumor cells) from inhibiting functions of an immune monitoring mechanism. In addition, when introduction target eukaryotic cells are bacteria-infected cells or virus-infected cells, it is preferable to use various polypeptides involved in induction of apoptosis of the infected cells, polypeptides that can prevent bacteria or viruses from proliferating in the infected cells, or polypeptides that can prevent bacterial or viral infection from spreading from the infected cells.

Here, like the carrier peptide fragment, the polypeptide as a foreign substance may contain a modified amino acid sequence formed by substituting, deleting and/or adding (inserting) one or several amino acid residues as long as its function is maintained.

The construct for introducing a foreign substance disclosed here is preferably a construct in which at least one amino acid residue is amidated. When carboxyl groups of amino acid residues (typically, C-terminal amino acid residues of a peptide chain) are amidated, it is possible to improve structural stability (for example, protease resistance) of peptide moieties in the construct for introducing a foreign substance.

For example, when a foreign substance is bound to the N-terminal side of the carrier peptide fragment, it is preferable to amidate the C-terminal amino acid residue of the carrier peptide fragment. In addition, for example, when the foreign substance is a polypeptide and the polypeptide is bound to the C-terminal side of the carrier peptide fragment, the C-terminal amino acid residue of the polypeptide can be amidated.

In the construct for introducing a foreign substance disclosed here, peptide moieties containing at least a carrier peptide fragment can be easily produced according to a general chemical synthesis method. For example, either a conventionally known solid-phase synthesis method or liquid-phase synthesis method may be used. A solid-phase synthesis method in which Boc (t-butyloxycarbonyl) or Fmoc (9-fluorenylmethoxycarbonyl) is applied as a protecting group for an amino group is preferable.

According to a solid-phase synthesis method using a commercially available peptide synthesizer, a peptide chain having a desired amino acid sequence and a modified (C-terminal amidation, etc.) moiety can be synthesized as the peptide moiety.

Alternatively, peptide moieties may be produced by biosynthesis based on a genetic engineering method. That is, a polynucleotide (typically, DNA) of a nucleotide sequence (including an ATG start codon) that encodes a desired amino acid sequence is synthesized. Then, a recombinant vector having an expression gene construct composed of a synthesized polynucleotide (DNA) and various regulatory elements (including a promoter, a ribosome binding site, a terminator, an enhancer, and various cis elements that control an expression level) for expressing the amino acid sequence in host cells is constructed according to host cells.

According to a general technique, this recombinant vector is introduced into predetermined host cells (for example, yeast, insect cells, and plant cells), and the host cells or tissues or subjects containing the cells are cultured under predetermined conditions. Accordingly, desired peptides can be produced in cells. Then, peptide moieties are isolated from the host cells (in a culture medium if secreted), and as necessary, refolding, purification, and the like are performed, and thereby a desired peptide moiety can be obtained.

Here, regarding a method of constructing a recombinant vector, a method of introducing a constructed recombinant vector into host cells, and the like, and methods conventionally used in the field may be directly used, and such methods themselves do not particularly characterize the present invention, and thus detailed description thereof will be omitted.

Alternatively, template DNA (that is, a synthetic gene fragment including a nucleotide sequence that encodes an amino acid sequence of a peptide moiety of a construct for introducing a foreign substance) for a cell-free protein synthesis system may be constructed, various compounds (ATP, RNA polymerase, amino acids, etc.) necessary for peptide moiety synthesis are used, and thus a desired polypeptide can be synthesized in vitro using a so-called cell-free protein synthesis system. Regarding the cell-free protein synthesis system, refer to, for example, the paper written by Shimizu et al. (Shimizu et al., Nature Biotechnology, 19, 751-755 (2001)), and the paper written by Madin et al. (Madin et al., Proc. Natl. Acad. Sci. USA, 97(2), 559-564 (2000)). Based on the techniques described in these papers, many companies had already commissioned polypeptides at the time of filing this application, and cell-free protein synthesis kits (for example, commercially available from CellFree Sciences Co., Ltd. Japan) are commercially available.

A single-stranded or double-stranded polynucleotide including a nucleotide sequence that encodes the peptide moiety of the construct for introducing a foreign substance disclosed here and/or a nucleotide sequence complementary to the sequence can be easily produced (synthesized) by conventionally known methods. That is, when codons corresponding to amino acid residues constituting a designed amino acid sequence are selected, a nucleotide sequence corresponding to the amino acid sequence is easily determined and provided. Then, once the nucleotide sequence is determined, a (single-stranded) polynucleotide corresponding to a desired nucleotide sequence can be easily obtained using a DNA synthesizer or the like. In addition, desired double-stranded DNA can be obtained using the obtained single-stranded DNA as a template according to various enzymatic synthetic techniques (typically, PCR). In addition, the polynucleotide may be in the form of DNA or in the form of RNA (mRNA, etc.). Double-stranded or single-stranded DNA may be provided. When single-stranded DNA is provided, it may be a coding strand (sense strand) or a non-coding strand (antisense strand) of sequence complementary thereto.

The polynucleotide obtained in this manner can be used as a material for constructing a recombinant gene (expression cassette) for peptide production in various host cells or a cell-free protein synthesis system as described above.

The construct for introducing a foreign substance disclosed here can be suitably used as an effective component of a composition for applications based on the function of the foreign substance. Here, the construct for introducing a foreign substance may be in a salt form as long as the function of the foreign substance is not lost. For example, an acid addition salt obtained by an addition reaction of an inorganic acid or organic acid that is generally used according to a general method can be used. Therefore, “construct for introducing a foreign substance” described in this specification and the claims includes such salt forms.

The composition of the construct for introducing a foreign substance disclosed here can contain various pharmaceutically (pharmacologically) acceptable carriers according to the usage form in addition to the construct for introducing a foreign substance as an effective component.

Regarding the carrier, for example, carriers that are generally used in the peptide drug as a diluent, an excipient, or the like are preferable. The carrier may appropriately vary depending on applications and forms of the construct for introducing a foreign substance, but typically, water, a physiological buffer solution, and various organic solvents may be exemplified. In addition, the carrier may be a non-drying oil such as an aqueous solution containing an alcohol (such as ethanol) with an appropriate concentration, glycerol, and an olive oil, or may be a liposome. In addition, examples of a secondary component that can be contained in the pharmaceutical composition include various fillers, extending agents, binders, moisturizers, surfactants, dyes, and fragrances.

The form of the composition is not particularly limited. Examples of typical forms include liquids, suspensions, emulsions, aerosols, foams, granules, powders, tablets, capsules, and ointments. In addition, for use in injection or the like, lyophilizates and granules for preparing a drug solution by performing dissolving in a saline or an appropriate buffer solution (for example, PBS) immediately before use can be provided.

A process itself of preparing various forms of drugs (compositions) including the construct for introducing a foreign substance (main component) and various carriers (secondary component) as materials may be performed according to a conventionally known method, and such a production method itself does not characterize the present invention, and thus detailed description thereof will be omitted. Examples of detailed sources of information on prescription include Comprehensive Medicinal Chemistry, edited by Corwin Hansch, published by Pergamon Press (1990).

Only a desired amount of the composition provided as described above in the form of a liquid can be administered to patients (that is, a living organism) through intravenous, intramuscular, subcutaneous, intradermal or intraperitoneal injection. Alternatively, a solid form such as a tablet can be administered orally. Accordingly, it is possible to obtain a desired activity in a living organism. Therefore, it is possible to improve pathological conditions in various diseases.

A method of introducing a construct for introducing a foreign substance, in a living organism (in vivo), or outside a living organism (in vitro) using the composition disclosed here (construct for introducing a foreign substance) is provided. The method roughly includes the following processes (1) to (3).

(1) a process of preparing a construct for introducing a foreign substance including a carrier peptide fragment composed of an amino acid sequence shown in SEQ ID NO: 1 and the desired foreign substance bound to an N-terminal side and/or C-terminal side of the carrier peptide fragment

(2) a process of supplying the construct for introducing a foreign substance into a sample containing desired (target) eukaryotic cells

(3) a process of incubating the sample to which the construct for introducing a foreign substance is supplied and introducing the construct into eukaryotic cells in the sample

The “eukaryotic cells” include, for example, various tissues, internal organs, organs, blood, lymph, and the like in vivo. The “eukaryotic cells” include, for example, various cell masses, tissues, internal organs, organs, blood, and lymph extracted from living bodies, and cell lines and the like in vitro.

The composition disclosed here can be used according to a method and in a dose depending on its form and purpose. For example, only a desired amount of the composition in the form of a liquid can be administered to affected parts (for example, malignant tumor tissues, virus-infected tissues, and inflamed tissues) of patients (that is, a living organism) through intravenous, intramuscular, subcutaneous, intradermal or intraperitoneal injection. Alternatively, a solid form such as a tablet or a gel-like or aqueous jelly-like form such as an ointment can be directly administered to predetermined tissues (that is, for example, an affected part such as tissues and organs including tumor cells, virus-infected cells, inflammatory cells, or the like). Alternatively, a solid form such as a tablet can be administered orally. In the case of oral administration, in order to prevent digestive enzyme decomposition in the digestive tract, encapsulation or a protective (coating) material is preferably applied.

Alternatively, with respect to eukaryotic cells cultured outside a living organism (in vitro), an appropriate amount of the composition disclosed here (that is, an appropriate amount of the construct for introducing a foreign substance) may be supplied to a culture solution containing desired eukaryotic cells at least once. The amount supplied each time and the number of times it is supplied are not particularly limited because they can vary depending on conditions such as the kind of eukaryotic cells to be cultured, the cell density (cell density when the culture starts), the number of passages, culture conditions, and the of the culture medium. For example, the composition is preferably added once, twice, or more times so that a concentration of the carrier peptide fragment in the culture solution is within a range of about 0.05 μM or more and 100 μM or less, for example, within a range of 0.5 μM or more and 50 μM or less.

Here, an example of the in vitro introduction method is shown in the following examples.

A method of evaluating introduction efficiency of the construct for introducing a foreign substance is not particularly limited. In such evaluation, for example, changes in the introduced cells (a morphological change, a change in the proliferation rate, etc.) may be observed. When a dye is bound to the carrier peptide fragment, the efficiency of introducing into eukaryotic cells can be evaluated using microscopic observation (for example, fluorescence microscopic observation), flow cytometry, or the like.

### EXAMPLES

While several examples of the present invention will be described below, these examples are not intended to limit the present invention.

### Example 1: Production of Construct for Introducing a Foreign Substance

Three synthetic peptides (Peptides 1 to 3) shown in Table 1 were prepared. Specifically, details are as follows.

Peptide 1 was a peptide composed of a carrier peptide fragment shown in SEQ ID NO: 1 disclosed here.

Peptide 2 was a peptide composed of NoLS of LIM kinase 2 (SEQ ID NO: 2).

Peptide 3 was a cell-penetrating peptide Pip6a disclosed in Non-patent Document 1 (SEQ ID NO: 3). Here, in the column “sequence” in Table 1, “X” indicates aminohexanoic acid. In the same column, “B” indicates β-alanine (the same applies to Table 2).

In both Peptide 1 and Peptide 2, a carboxyl group (—COOH) of a C-terminal amino acid was amidated (—CONH₂). Both Peptide 1 and Peptide 2 were synthesized by performing a solid-phase synthesis method (Fmoc method) using a commercially available peptide synthesizer according to the manual. Peptide 3 was a commercial product (catalog number: crb1000352h, commercially available from Cambridge Research Biochemicals).

Here, since a manner of use of the peptide synthesizer itself does not characterize the present invention, detailed description thereof will be omitted.

TABLE 1

| Peptide No. | Sequence | SEQ ID NO |
|---|---|---|
| 1 | TLKERCLQVVRSLVKKKRTLRKNDRKKR | 1 |
| 2 | KKRTLRKNDRKKR | 2 |
| 3 | RXRRBRRXRYQFLIRXRBRXRB | 3 |

The amino acid residues on the N-terminal side of Peptide 1 and Peptide 2 and the amino acid residue on the C-terminal side of Peptide 3 were bound to a fluorescent dye as the foreign substance. In this manner, three constructs for introducing a foreign substance were produced. Specifically, FAM, which is a general fluorescent dye (that is, C₂₁H₁₂O₇: 5(6)-Carboxyfluorescein, a molecular weight of 376.3, an excitation wavelength of 495 nm, and a fluorescence wavelength of 520 nm) was directly bound to the N-terminal side of Peptide 1 and Peptide 2 and the C-terminal side of Peptide 3 based on a general method, and constructs for introducing a foreign substance based on Peptides 1 to 3 were produced.

Table 2 shows the configuration of the construct for introducing a foreign substance based on Peptide 1. Hereinafter, this will be referred to as Sample 1.

Table 2 shows the configuration of the construct for introducing a foreign substance based on Peptide 2. Hereinafter, this will be referred to as Sample 2.

Table 2 shows the configuration of the construct for introducing a foreign substance based on Peptide 3. Hereinafter, this will be referred to as Sample 3.

The samples obtained in this manner were diluted with DMSO to prepare a total of three sample solutions (stocks) having a sample concentration of 2 mM.

TABLE 2

| Sample No. | Configuration |
|---|---|
| 1 | FAM-TLKERCLQVVRSLVKKKRTLRKNDRKKR-CONH₂ |
| 2 | FAM-KKRTLRKNDRKKR-CONH₂ |
| 3 | RXRRBRRXRYQFLIRXRBRXRB-FAM |

### Example 2: Evaluation of Cell-Penetrating Function of Samples 1 to 3

HeLa cells (established cell lines derived from human cervical cancer cells) were used as eukaryotic cells, and cell-penetrating functions of three samples (constructs for introducing a foreign substance) obtained in Example 1 were examined

In this example, Examples 1 to 3 were set according to the kind of peptide used as shown below. In addition, as Examples 4 and 5, peptide-free groups were set. The following test was performed in a total of 5 categories. Here, the concentration described below was a final concentration during the following 20-hour incubation.

### Example 1: Sample 1, 10 μM

### Example 2: Sample 2, 10 μM

### Example 3: Sample 3, 10 μM

Example 4: No sample (peptide) added, FAM, 10 μM
Example 5: No sample (peptide) added

Here, in all of the examples, the concentration of DMSO was adjusted to 0.5% during the following 20-hour incubation. In addition, the number of test wells (n) in each of the above examples was set to 3.

### Example 1

HeLa cells were cultured using a mixed solution containing a 90% Eagle's MEM medium (containing 0.1% non-essential amino acid, 2 mM of L-glutamine, 1 mM of sodium pyruvate, and 1.5 g/L of sodium bicarbonate) and 10% serum (FBS: fetal bovine serum) as a culture solution.

The HeLa cells were subjected to a trypsin treatment with a 0.25% trypsin/EDTA solution at 37° C. for 3 minutes. After the above treatment, trypsin was inactivated using the culture solution. Centrifugation was performed and the centrifugal supernatant was removed. The cell pellet obtained by centrifugation was suspended in a fresh culture solution. A cell suspension (sample for introducing a foreign substance) having a cell concentration of about 1×10⁵ cells/well was prepared from the culture solution, and 2 ml thereof was seeded in each well of a commercially available 6-well plate (commercially available from Iwaki). Then, the cells were cultured at 37° C. for 4 hours under 5% CO₂ conditions, and adhesion of the seeded cells was confirmed.

Then, 1 ml of the culture supernatant was removed from the well after the culture for 4 hours, and a culture solution containing Sample 1 at a concentration of 20 μM was added. That is, the final sample concentration in the well was 10 μM.

The plate was then incubated at 37° C. for 20 hours under 5% CO₂ conditions.

After such incubation for 20 hours, the cells were washed twice with PBS at 1 ml/well. Then, the cells in the wells were treated with a 0.25% trypsin/EDTA solution at 200 μL/well at 37° C. for 3 minutes. Then, a culture solution at 400 μL/well was additionally added and the cells were collected in the tube. Then, the wells were additionally washed with PBS at 600 μL/well, and the cells remaining in the wells were collected in the above tube. The tube was centrifuged at 4° C. for 5 minutes under 210×g conditions. The centrifugal supernatant was removed, and the cell pellet was suspended (washed) in 1 ml PBS, and centrifuged under the same conditions as above. This operation was repeated twice.

Then, the collected cells were analyzed using a flow cytometer. On-Chip Flowcytometer (commercially available from On-Chip Biotechnologies Co., LTD.) was used as the flow cytometer.

In such analysis, the cells obtained as described above were suspended in 50 μL of PBS. 50 μL of 2×sample buffer for the above flow cytometer was additionally added to this suspension to prepare a cell suspension for analysis.

Using the above flow cytometer, gating was performed based on forward scatter (FSC) and side scatter (SSC), and a gate was set for a measurement target cell population. Then, the fluorescent intensity of the cell population within such a gate was measured. The fluorescent intensity of 10,000 measurement target cells was measured for each measurement sample. For measurement, a laser lamp FL2 of the flow cytometer was used. The measurement results were analyzed using commercially available analysis software “FlowJo (registered trademark)” (commercially available from TreeStar), and a mean fluorescent intensity (MFI) of measurement target cell populations was obtained.

### Examples 2 and 3

Examples 2 and 3 were performed using the same materials and processes as in Example 1 except that Sample 2 (Example 2) or Sample 3 (Example 3) was used instead of Sample 1.

### Example 4

In this example, Example 4 was performed using the same materials and processes as in Example 1 except that no sample was added, and FAM was added at the above concentration.

### Example 5

In this example, Example 5 was performed using the same materials and processes as in Example 1 except that neither the sample nor FAM was added.

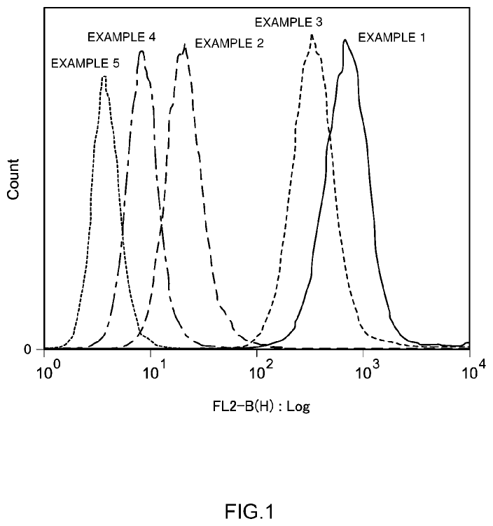

The results obtained in Examples 1 to 5 are shown in FIG. 1 and Table 3. FIG. 1 shows typical data (histogram) obtained by the flow cytometry. Table 3 shows the MFI corresponding to the histogram in FIG. 1.

TABLE 3

| | Exam- | Exam- | Exam- | Exam- | Exam- |
|---|---|---|---|---|---|
| | ple 1 | ple 2 | ple 3 | ple 4 | ple 5 |
| MFI | 8.2 × 10² | 24 | 4.0 × 10² | 9.7 | 4.2 |

As shown in FIG. 1 and Table 3, when Examples 1 to 4 which are groups in which a fluorescent dye (FAM) was added were compared with Example 5 in which no fluorescent dye was added, the peaks of the histogram were shifted to the X axis in the right direction (refer to FIG. 1). In addition, as shown in Table 3, it was confirmed that the MFI of Examples 1 to 4 was larger than the MFI of Example 5. In addition, comparing Examples 1 to 3 with Example 4, it was found that the peak shift of the histogram and the degree of increase in MFI were further increased due to addition of the sample. That is, it was confirmed that introduction of a fluorescent dye (FAM) into cells was improved due to addition of Samples 1 to 3. Here, it was confirmed that, since the MFI of the cell sample to which Sample 1 was added was detected at the highest, the cell permeability of Sample 1 was the best.

It was confirmed that, among three samples (construct for introducing a foreign substance) obtained in Example 1, the construct for introducing a foreign substance according to Sample 1 contained a carrier peptide fragment composed of an amino acid sequence shown in SEQ ID NO: 1, and thus it exhibited excellent cell permeability. In addition, according to the examination by the inventors, it was confirmed that, regardless of whether the foreign substance was not only a fluorescent dye but also any of a polypeptide, a nucleic acid, and a drug, the foreign substance was efficiently introduced from the outside of the cells into the cytoplasm through the cell membrane.

As clearly understood from above, according to the technology disclosed here, as a particularly preferable aspect of the method of introducing a foreign substance, there is provided a method of introducing a desired foreign substance from the outside of eukaryotic cells into at least the cytoplasm of the cells in vitro, which is a method using a carrier peptide fragment composed of SEQ ID NO: 1. Such a carrier peptide fragment is suitable to introduce a desired foreign substance into the cytoplasm.

While specific examples of the technology disclosed here have been described above in detail, these are only examples, and do not limit the scope of the invention. The technologies described in the claims of the invention include various modifications and alternations of the above exemplified specific examples.

According to the technology disclosed here, there is provided a construct artificially produced in order to introduce a desired foreign substance from the outside of eukaryotic cells (particularly, various animal cells having no cell wall represented by humans and other mammals) into the cytoplasm. When such a construct is used, a desired foreign substance can be effectively introduced into desired cells, cells into which the foreign substance is introduced, and organs and other living tissues including cells containing the foreign substance can be obtained. In addition, when such a construct is used, a treatment strategy for diseases can be provided.

1. A method of introducing a foreign substance from the outside of eukaryotic cells into at least a cytoplasm of the cells in vitro or in vivo,
the method comprising the following steps:
(1) preparing a construct for introducing a foreign substance, wherein the construct includes a carrier peptide fragment composed of the following amino acid sequence:
TLKERCLQVVRSLVKKKRTLRKNDRKKR (SEQ ID NO: 1), and
the foreign substance bound to an N-terminal side and/or C-terminal side of the carrier peptide fragment;
(2) supplying the construct into a sample containing target eukaryotic cells; and
(3) incubating the sample to which the construct is supplied, wherein the construct is introduced into the target eukaryotic cells in the sample.

2. The method according to claim 1, wherein the foreign substance is any organic compound selected from the group consisting of polypeptides, nucleic acids, dyes and drugs.

3. The method according to claim 2, wherein
the foreign substance is a mature polypeptide derived from any biological species or its precursor polypeptide,
and
the construct is a synthetic polypeptide having an amino acid sequence corresponding to a mature polypeptide or its precursor polypeptide as the foreign substance, and an amino acid sequence of the carrier peptide fragment.

4. The method according to claim 3, wherein the amino acid sequence corresponding to a mature polypeptide or its precursor polypeptide as the foreign substance is arranged on the N-terminal side of the carrier peptide fragment.

5. The method according to claim 1, wherein the target eukaryotic cells to which the construct is introduced are human or non-human mammal cells.

6. A construct for introducing a foreign substance from the outside of eukaryotic cells into at least a cytoplasm of the cells, comprising
a carrier peptide fragment composed of the following amino acid sequence:
TLKERCLQVVRSLVKKKRTLRKNDRKKR (SEQ ID NO: 1), and
a foreign substance bound to an N-terminal side and/or C-terminal side of the carrier peptide fragment.

7. The construct according to claim 6, wherein the foreign substance is any organic compound selected from the group consisting of polypeptides, nucleic acids, dyes and drugs.

8. The construct according to claim 7, wherein
the foreign substance is a mature polypeptide derived from any biological species or its precursor polypeptide,
and
the construct is a synthetic polypeptide having an amino acid sequence corresponding to a mature polypeptide or its precursor polypeptide as the foreign substance and an amino acid sequence of the carrier peptide fragment.

9. The construct according to claim 8, wherein the amino acid sequence corresponding to a mature polypeptide or its precursor polypeptide as the foreign substance is arranged on the N-terminal side of the carrier peptide fragment.
